(19) United States
(12) Patent Application Publication
(10) Pub. No.: US 2008/0312704 A1
(43) Pub. Date: Dec. 18, 2008

(54) INSTRUMENTATION AND ASSOCIATED TECHNIQUES FOR MINIMALLY INVASIVE SPINAL CONSTRUCT INSTALLATION

(75) Inventors: Hugh D. Hestad, Edina, MN (US); W. Matthew Kuester, St. Louis Park, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: ZIMMER SPINE, INC., Minneapolis, MN (US)

(21) Appl. No.: 11/761,404

(22) Filed: Jun. 12, 2007

(51) Int. Cl. A61F 5/00; A61B 17/70

(52) U.S. Cl. 606/86 A; 606/246

(57) ABSTRACT

A spinal fixation installation assembly includes an elongate member having a trailing end. The elongate member, which could be a rigid member, is configured for travel in a first direction along an elongate portion of a first bone anchor coupled to a spine. The elongate member is also configured to travel in a second direction between the first bone anchor and a second bone anchor also coupled to the spine. A cord member is coupled to the trailing end of the elongate member. In one aspect of this embodiment, a spine rod is coupled to the cord member at its leading end and is configured for travel in the first and second directions. The elongate portion of one or more of the bone anchors may extend percutaneously from the spine, while the spine rod may be releasably coupled to the cord member.

### FIELD OF THE INVENTION

This invention relates generally to spinal fixation surgery and more specifically relates to instrumentation and associated techniques for minimally invasive installation of vertebral connecting elements of spinal fixation constructs.

### BACKGROUND

The spinal column is a highly complex system of bones and connective tissues that provides support for the body and protects the delicate spinal flexible connecting member and nerves. The spinal column includes a series of vertebrae stacked one on top of the other, each vertebral body including an inner or central portion of relatively weak cancellous bone and an outer portion of relatively strong cortical bone. An intervertebral disc is situated between each vertebral body to cushion and dampen compressive forces experienced by the spinal column. A vertebral canal containing the spinal cord and nerves is located posterior to the vertebral bodies. In spite of the complexities, the spine is a highly flexible structure, capable of a high degree of curvature and twist in nearly every direction. For example, the kinematics of the spine normally includes flexion, extension, rotation and lateral bending.

There are many types of spinal column disorders including scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease, or trauma, such as ruptured or slipped discs, degenerative disc disease, fractured vertebra, and the like. Patients that suffer from such conditions usually experience extreme and debilitating pain as well as diminished range of motion and nerve function. These spinal disorders may also threaten the critical elements of the nervous system housed within the spinal column.

One particular spinal fixation technique includes immobilizing the spine by using connecting elements or orthopedic spine rods that run generally parallel to the spine. This is accomplished by exposing the spine posterially and fastening hooks, bone screws, or anchors to the pedicles of the appropriate vertebrae. The vertebral anchors are generally placed two per vertebrae, one at each pedicle on either side of the spinal column and serve as anchor points for the connecting elements or spine rods. The aligning influence of the rods forces the spine to conform to a more desirable shape. In many cases, the spine rods are bent to achieve the desired curvature of the spinal column.

Installation of such spinal fixation constructs conventionally requires a surgeon to prepare a long incision aligned with the spinal column of a patient. The pedicle screws, hooks or other vertebral anchors are then attached to a number of vertebrae after which the connecting element or spine rod is located with respect to saddles or U-shaped channels attached to the vertebral anchors. Conventional surgical methods require a large midline incision and retraction of skin, muscle and other tissue to provide the surgeon with sufficient visualization of the pedicle bone structure.

The accuracy of the placement and configuration of the spinal fixation elements are very important. In combination with the relatively long incision typically required for the installation of the fixation construct, extended surgical procedures and related difficulties may lead, for example, to extended patient recovery. Therefore, surgical techniques and the associated instrumentation to accomplish more minimally invasive installation of spinal fixation constructs are highly desirable to avoid the problems associated with known surgical installation techniques.

### SUMMARY OF THE INVENTION

This invention addresses these and other shortcomings in the prior art. The devices and methods associated with this invention are used to aid in the surgery and installation of vertebral fixation components, particularly the connecting element or spinal rod.

In known spinal fixation systems vertebral anchors such as pedicle screws are inserted into the target vertebrae of a patient's spinal column. The spinal fixation system may include a connecting element joining at least two vertebral anchors to provide added support and a degree of rigidity to the patient's spine. The connecting element may be a rigid spine rod that is generally linear or otherwise suitably shaped, or may alternatively be a less rigid structure. Nevertheless, installation of the connecting element to the vertebral anchors coupled to the respective vertebrae is facilitated through a minimally invasive surgical procedure according to various embodiments of this invention.

In one aspect, this invention is directed to a spinal fixation installation assembly including an elongate member having a trailing end. The elongate member, which could be a rigid member, is configured for travel in a first direction along an elongate portion of a first bone anchor coupled to a spine. The elongate member is also configured to travel in a second direction between the first bone anchor and a second bone anchor also coupled to the spine.

A cord member is coupled to the trailing end of the elongate member. In one aspect of this embodiment, a connecting element or spine rod is coupled to the cord member at its leading end and is configured for travel in the first and second directions.

In various embodiments, the elongate portion of one or more of the bone anchors may extend percutaneously from the spine, while the spine rod may include a taper adapted to facilitate travel of the rod between the first and second bone anchors. The spine rod may be releasably coupled to the cord member.

The spinal fixation installation assembly may also include a gripper member coupled to the cord member and adapted to pull the cord member in a third direction. The gripper member is, in one embodiment, further coupled to a percutaneous portion of the second bone anchor.

In another embodiment, a spinal fixation system includes first and second adjacent bone anchors coupled to a spine. The bone anchors, which may be in the form of pedicle screw assemblies, respectively include lumens adapted to extend percutaneously from the spine. A spine rod is adapted for fixation against the bone anchors. In one aspect of this embodiment, a cord member is coupled to a leading end of the spine rod, while an elongate member such as a rigid member is coupled to the cord member. The elongate member, thus, can guide the cord member within each of and between the lumens, thereby guiding the spine rod within the first lumen and between the two lumens. The elongate portion may be coupled to a base portion of the bone anchor along a frangible joint, such that separation of the two portions is thereby facilitated.

In yet another embodiment, a method of implanting a spine rod between two bone anchors coupled to a spine includes guiding an elongate member coupled to a spine rod in a first direction within a first elongate portion of the first bone anchor. The first direction is generally toward the spine of the patient. The elongate member is then guided in a second direction toward a second bone anchor, and in a third direction within a second elongate portion of the second bone anchor. The third direction is generally away from the spine. In one aspect of this embodiment, guiding of the elongate member induces travel of the spine rod in the first and second directions.

The various embodiments of this invention enable the surgeon to install the spinal fixation construct with smaller discrete incisions as opposed to an extended incision. As such, a more minimally invasive surgical procedure can be accomplished with this invention thereby promoting post-surgery patient recovery. As a result of these and other aspects of this invention, increased efficiency and accuracy is provided for installation of a spinal fixation construct in a minimally invasive atmosphere thereby promoting patient recovery and optimum spinal surgery results.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a side elevational and partial cross-sectional view of a spinal fixation system being surgically implanted in selected vertebrae of a patient's spine according to one embodiment of this invention;

FIG. 2 is a side elevational and partially cross-sectional view of the embodiment of FIG. 1 showing a subsequent step in the implantation of the spinal fixation system;

FIG. 3 is a side elevational and partially cross-sectional view of the embodiment of FIGS. 1-2 showing a further subsequent step in the implantation of the spinal fixation system, including a gripper member;

FIG. 4 is a side elevational and partially cross-sectional view of the embodiment of FIGS. 1-3 showing a further subsequent step in the implantation of the spinal fixation system, including a spine rod;

FIG. 5 is a side elevational and partially cross-sectional view of the embodiment of FIGS. 1-4 showing a final step in the implantation of the spinal fixation system;

FIG. 6 is a side elevational and partially cross-sectional view of an alternative embodiment of the gripper member of FIG. 3;

FIG. 7 is a side elevational and partially cross-sectional view of another embodiment of a spinal fixation system being surgically implanted in selected vertebrae of a patient's spine; and

FIG. 8 is a side elevational and partially cross-sectional view of the embodiment of FIG. 7 showing a subsequent step in the implantation of the spinal fixation system thereof.

### DETAILED DESCRIPTION OF THE INVENTION

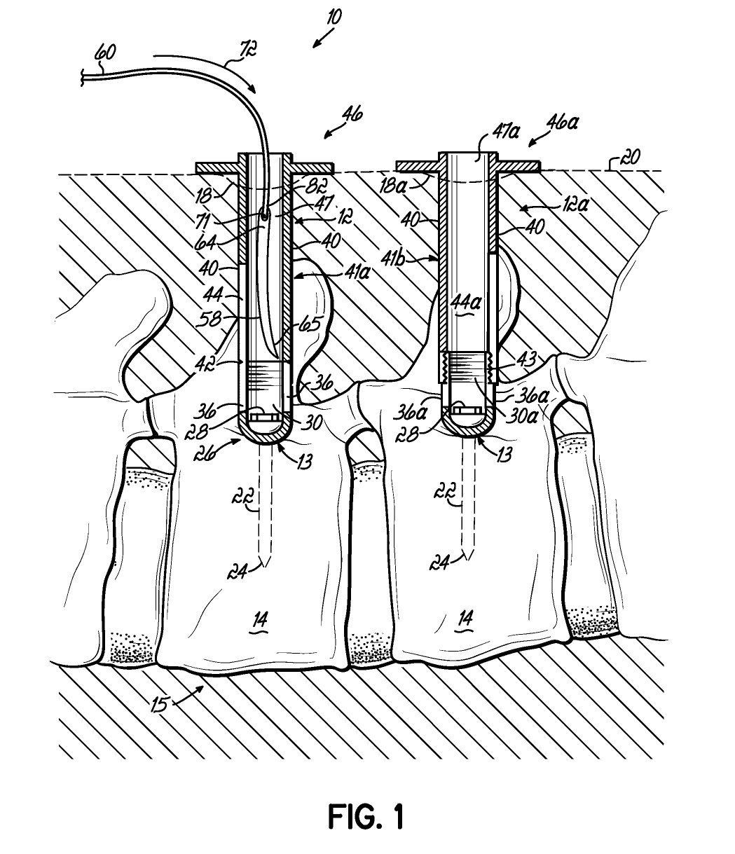

Referring to the drawings, various components and tools are shown to enable a minimally invasive surgery to install a spinal fixation system. In FIGS. 1-5, an exemplary spinal fixation system 10 includes a number of bone anchors 12, 12a that, in one embodiment, are each pedicle screw assemblies, each of which is inserted into selected vertebrae 14 of a patient. The pedicle screw assemblies 12, 12a are joined together in the spinal fixation system by a connecting element which in one embodiment is a spine rod 16. The connecting element may be a rigid rod or alternatively something other than a rigid rod, such as a flexible connecting element. For ease of understanding, the connecting member is hereinafter referred to as “spine rod 16.”

Each of the pedicle screw assemblies 12, 12a may be inserted into the patient through discrete and often individual incisions 18 in the patient's skin 20. In certain instances, a single incision 18 may be available to provide installation of multiple pedicle screw assemblies 12 in adjacent vertebrae 14 of a spinal column (hereinafter “spine 15”). The small, discrete incisions 18 provide the opportunity for insertion of a cannulated pedicle screw via a K-wire inserted through the incision 18 to the precise location on the vertebrae 14 for proper installation of the pedicle screw assembly 12. While cannulated and other types of pedicle screws are contemplated and described herein, one of ordinary skill in the art will appreciate that other types of bone anchors and vertebrae engaging mechanisms can be utilized such as hooks for anchoring the spine rod 16 to the patient's spine 15.

With continued reference to FIGS. 1-5, the pedicle screw assembly 12 includes a bone anchor, such as a pedicle screw 13, having a threaded shaft 22 and a distal tip 24 for insertion and stable positioning into the pedicle area of the patient's vertebrae 14. The exemplary pedicle screw assembly 12 shown herein is a polyaxial pedicle screw in which a base portion in the form of a polyaxial body 26 mounted opposite from the distal tip 24 of the pedicle screw assembly 12 to a screw head 28 provides for a variety of orientations of the polyaxial body 26 relative to a longitudinal axis of the threaded shaft 22 of the screw assembly 12 as is common with many pedicle screw systems. The polyaxial body 26 is coupled to the pedicle screw head 28 and includes a saddle or U-shaped lateral channel 30 formed between a pair of spaced arms 36 extending upwardly. The polyaxial body 26 is adapted to receive the spine rod 16 in the lateral channel 30 and the spine rod 16 is securely retained by the polyaxial body 26 of the pedicle screw 13 via a fastener such as a set screw 34 threadably received therein as is common with many known pedicle screw systems.

While the exemplary embodiment of FIGS. 1-5 is described including pedicle screw assemblies 12 having each a polyaxial body 26, pedicle screw assemblies each having a uniaxial body are also contemplated.

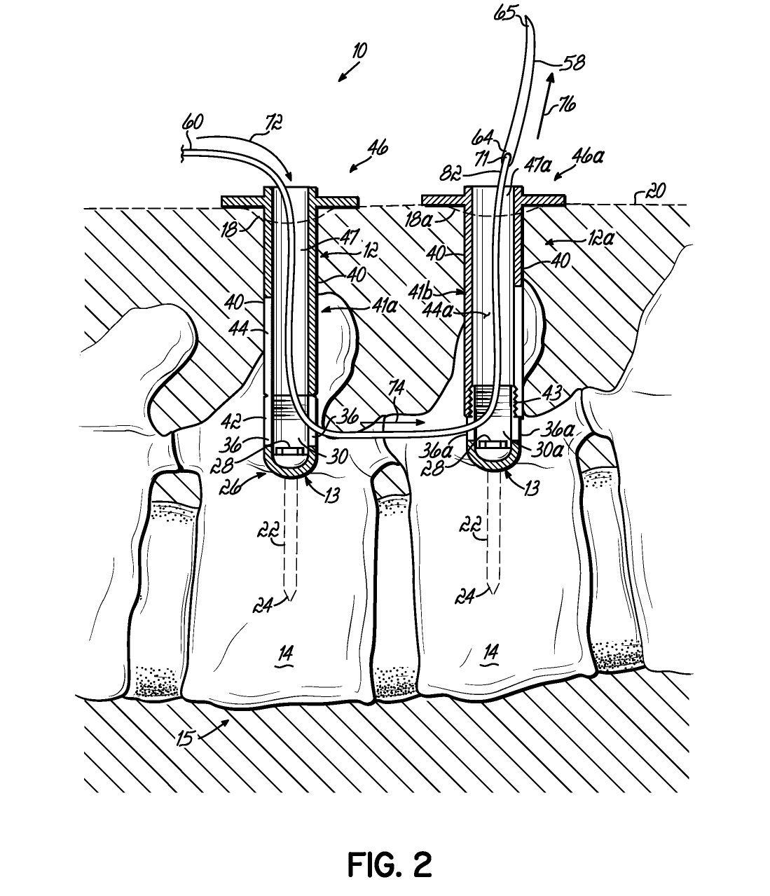

In one aspect of the embodiment of FIGS. 1-5, each pedicle screw assembly 12 includes a pair of tabs 40 extending upwardly from the polyaxial body 26, thereby defining an elongate portion of the pedicle screw assembly 12. Advantageously, the tabs 40 are extended to project through the incision 18 such that a distal end of the tabs 40 is located percutaneously above the patient's skin 20 when the threaded shaft 22 of the pedicle screw assembly 12 is inserted into the vertebrae 14 as shown in FIG. 2.

In this illustrative embodiment, the tabs 40 are generally arcuate and are each coupled to one of the arms 36 of the polyaxial body 30. The arcuate shape and position of the tabs 40 of the elongate portion of the pedicle screw assembly 12 thereby define a percutaneously-extending tubular portion thereof. For ease of understanding, two contemplated types of coupling between the arms 36 and tabs 40 are depicted in FIGS. 1-5. The exemplary pedicle screw assembly 12 (on the left side of each of FIGS. 1-5), for example, includes coupling between each of the arms 36 and one of the tabs 40 via a reduced thickness frangible joint 42. The exemplary pedicle screw assembly 12a (on the right side of each of FIGS. 1-5) includes a threaded coupling 43 between the arms 36 and tabs 40. Any other suitable type of coupling between the arms 36 and tabs 40 is similarly contemplated including, for example, and without limitation, magnetic components, adhesives or the like. In the embodiment shown, the tabs 40 form tubular structures 41a, 41b that provide percutaneous access to the pedicle screws 13. The tubular structures 41a, 41b can have any desired shape, such as round, oval, square, and rectangular.

While FIGS. 1-5 each depict two adjacent pedicle screw assemblies 12, 12a having two different types of coupling between the arms 36 and tabs 40, as described above, those of ordinary skill in the art will readily appreciate that any two adjacent pedicle screw assemblies can have the same or different types of coupling. For ease of understanding, the remainder of the description hereinafter generally refers to pedicle screw assembly 12 although the principles thereof are applicable to other pedicle screw assemblies such as the illustrative pedicle screw assembly 12a.

With continued reference to FIGS. 1-5, at least one of the tabs 40 of each pedicle screw assembly 12 is shaped to define a slot 44 in communication with at least one of the lateral channels 30 to permit, as described below, reorientation of a rigid member 58 and the spine rod 16. Each pedicle screw assembly 12 further includes a flange 46 formed proximate the proximal ends of the tabs 40. The flange 46 may have a generally rectangular or square configuration and be adapted to be juxtaposed on top of the patient's skin 20 at the associated incision 18. The flange 46 provides added stability to the construct as well as a platform for additional devices such as a light attachment (not shown) to increase the visualization of the surgical site. Other examples of pedicle screw assemblies are available in U.S. patent application Ser. No. 11/558,060 filed Nov. 9, 2006, the disclosure of which is herein incorporated by reference in its entirety.

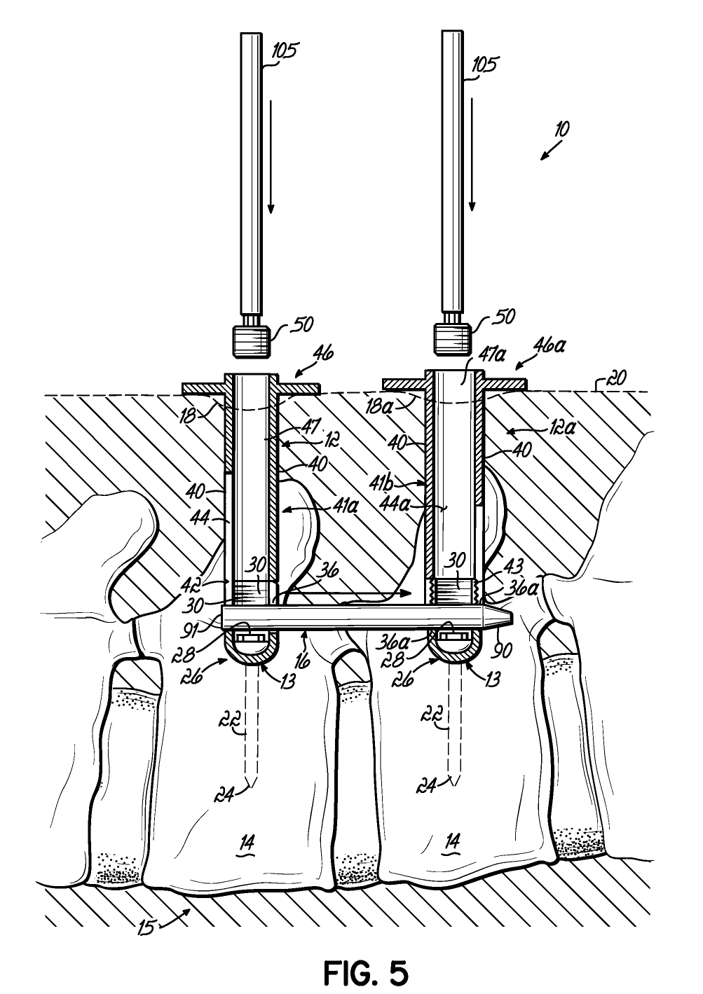

In one aspect of the embodiment of FIGS. 1-5, and with particular reference to FIG. 5, various components may be inserted through a lumen 47 formed between the tabs 40. For example, a set screw 50 may be inserted through the lumen 47 for mating with cooperating portions of the pedicle screw assembly 12, thereby securing the spine rod 16 within the polyaxial body 26 of the pedicle screw 13. Advantageously, the lumen 47 further allows insertion of a rod installation assembly, as explained below.

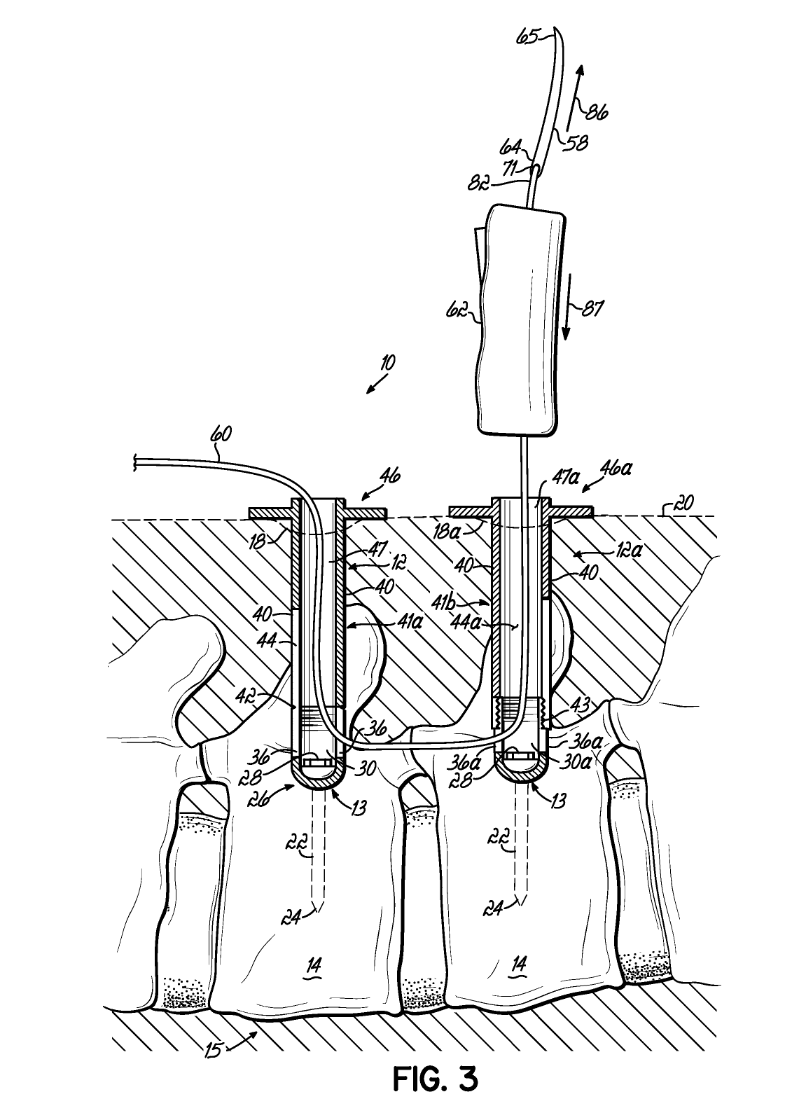

With reference to FIGS. 1-3, the rod installation assembly includes a rigid member 58, such as a needle, suitably coupled to a flexible string or cord member 60, the spine rod 16 and a gripper member in the form of a handle 62. The rigid member 58 can be shaped to more easily pass through human tissue. With particular reference to FIG. 3, the rigid member 58 defines a leading end of the rod installation assembly 52. The rigid member 58 is suitably dimensioned to fit within the lumen 47 such that it can be maneuvered within the lumen 47 and such that a surgeon can handle it. To this end, the rigid member 58 may have a suitable length such that a surgeon can easily guide the rigid member 58 within the lumen 47. Moreover, a trailing end 64 of the rigid member 58 is designed to be conveniently gripped, for example, by two human fingers of a surgeon carrying on the surgical procedure.

The rigid member 58 may be shaped such that reorientation and passage of the rigid member 58 through a lateral channel 30 of the polyaxial body 26 can be achieved with relative ease. To this end, the rigid member 58 may include a generally arcuate shape, as depicted in FIGS. 1-3. Reorientation of the rigid member 58 from a first direction in which the rigid member 58 travels within the first lumen 47 toward the spine 15 to a second direction in which it travels toward the second pedicle screw assembly 12a is further facilitated by the slot 44 of at least one of the tabs 40. More particularly, the slot 44 provides a space that receives the trailing end 64 of the rigid member 58 as the rigid member 58 turns from the first to the second direction.

Similarly, a slot 44a on the pedicle screw assembly 12a further facilitates reorientation of the rigid member 58. More particularly, the slot 44a provides a space that receives the leading end 65 of the rigid member 58 as the rigid member 58 turns from the second direction to a third direction up the lumen 47a.

As mentioned above, the rigid member 58 is suitably coupled to the cord member 60. Such coupling is chosen such that it may sustain tension applied by the rigid member 58 as the rigid member 58 is guided into the lumen 47 and through the lateral channels 30, 30a corresponding to two adjacent pedicle screw assemblies 12, 12a. Coupling of the rigid member 58 and cord member 60 may include any suitable type of coupling known to those of ordinary skill in the art. For example, and without limitation, such coupling may include an aperture 71 located in the trailing end 64 of the rigid member 58, and adapted to receive the cord member 60 there through. Moreover, a knot (not shown) or other suitably chosen arrangement may restrict the cord member 60 to prevent disengagement thereof from the rigid member 58. Alternatively, the rigid member 58 and member 60 may be formed as a single unit.

With continued reference to FIGS. 1-3, the rigid member 58 is made of a suitably chosen material and structure such that it may resist fracture otherwise caused by contact with surfaces within the lumen 47 or lateral channels 30, 30a. To this end, the rigid member 58 may, for example, be of unitary metallic structure. Other considerations for choice of the rigid member 58 include the degree of biocompatibility thereof. For example, the rigid member 58 may include titanium, a titanium alloy, or any other metal conventionally used for surgical procedures.

While FIGS. 1-3 depict a rigid member 58 having the shape, dimensions and coupling components as shown, those of ordinary skill in the art will readily appreciate that the rigid member 58 may take on any other of such characteristics, so long as they meet the generally suggested requirements described above.

As explained above, the rigid member 58 is coupled to and guides travel of the cord member 60. As such, the cord member 60 generally includes a flexible structure and materials such that it may easily travel through both of the lumens 47, 47a, lateral channels 30, 30a, and human tissue there between (not shown). Choice for materials defining the cord member 60, thus, may include considerations such as biocompatibility, tensile strength, modulus of elasticity and coefficient of friction against surfaces of the lumens 47, 47a, lateral channels 30, 30a and surrounding tissue. For example, and without limitation, the cord member 60 may be in the form of a relatively thin nickel-titanium alloy wire, a stainless steel coiled wire, or a polymer-based material. The degree of flexibility of the cord member 60 may be such, for example, that it may bend as depicted by the arrow 72 and the general shape of the cord member in FIGS. 3-5.

With reference to FIGS. 1-2, the rigid member 58 and cord member 60 are guided in the direction depicted by arrow 72 (i.e., toward the spine 15), turned about 90 degrees through the lateral channel 30 and guided in a second direction through tissue between the two adjacent pedicle screw assemblies 12, 12a, generally as indicated by arrow 74. Subsequently, the rigid member 58 is inserted through the lateral channel 30a of the pedicle screw assembly 12a, turned about 90 degrees and guided in a third direction up the lumen 47a, as generally indicated by the arrow 76 (i.e., away from the spine 15).

With reference to FIGS. 2-3, once the rigid member 58 has been guided away from the skin 20, a construct is achieved wherein the cord member 60 extends between the two adjacent incisions 18, 18a and through the respective lumens 47, 47a and lateral channels 30, 30a.

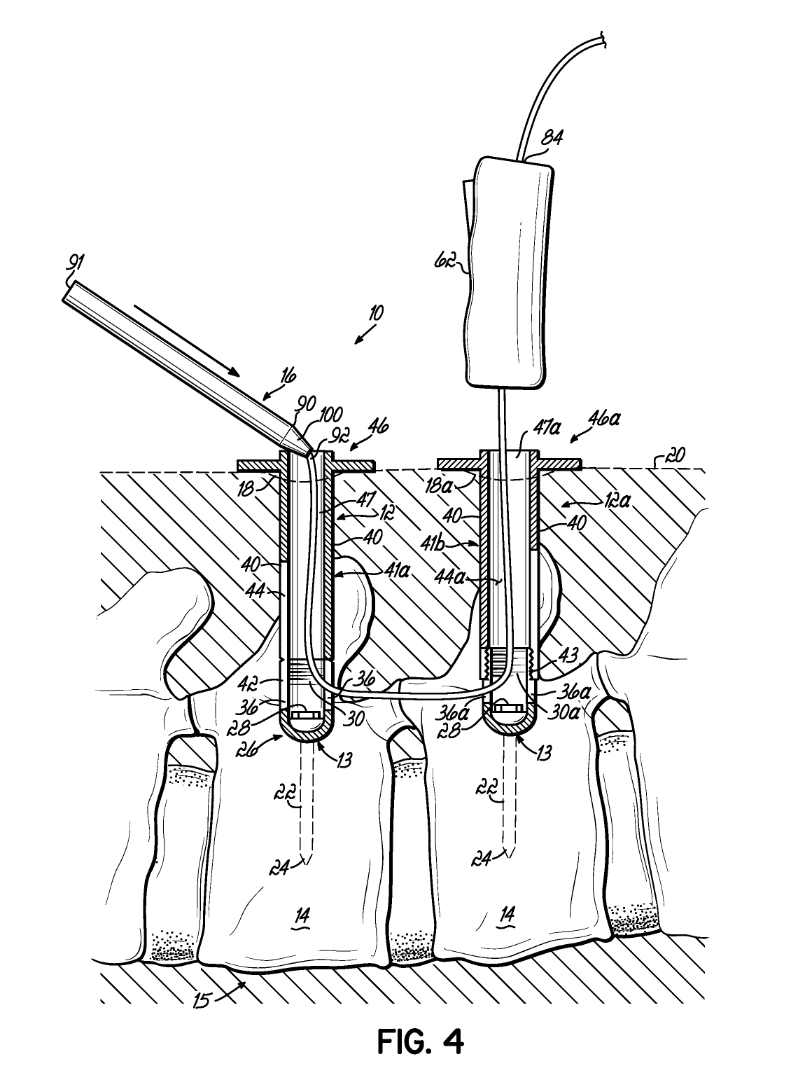

In another aspect of the embodiment of FIGS. 1-5, and more particularly with reference to FIGS. 3-4, a gripper member in the form of a handle 62 may be coupled to the leading end 82 of the cord member 60 to facilitate gripping and pulling thereof. Any suitable type, dimensions and configurations are contemplated for the handle 62. For example, and without limitation, the handle 62 may be of a type that requires guiding the rigid member 58 and leading end 82 of the cord member 60 through a bore 84 there through, generally as indicated by the movement of arrows 86, 87 relative to one another. In this exemplary embodiment, the handle 62 is deformable such that it may be compressed to frictionally engage the cord member 60. Once frictionally engaged with the cord member 60, the handle 62 can be pulled to thereby pull the cord member 60 therewith. Alternatively, the handle 62 may include an actuator 88 such that actuating thereof effects pulling of the cord member 60.

Other alternative gripper members (not shown) may include non-frictional engagement with the cord member 60, and may further require, for example, the formation of knots to define such engagement. Other means to facilitate such engagement may include, without limitation, adhesives, chemical or mechanical bonding, mechanical fasteners and/or magnetic components. Likewise, alternative handles may include a slot to replace the bore 84 of the exemplary handle 62.

With reference to FIGS. 4-5, the rod installation assembly 52 includes a spine rod 16 that is ultimately positioned between two adjacent pedicle screw assemblies 12, 12a, such that the spine rod 16 extends through the respective lateral channels 30, 30a thereof. The spine rod 16 is coupled, at its leading end 90, to the trailing end portion 92 of the cord member 60 such that the cord member 60 can guide the spine rod 16 into the position described above. To this end, coupling of the spine rod 16 to the cord member 60 is such that the coupling can sustain tension applied as the cord member 60 is pulled through the lumen 47, through tissue and between the two adjacent lateral channels 30, 30a of the respective pedicle screw assemblies 12, 12a.

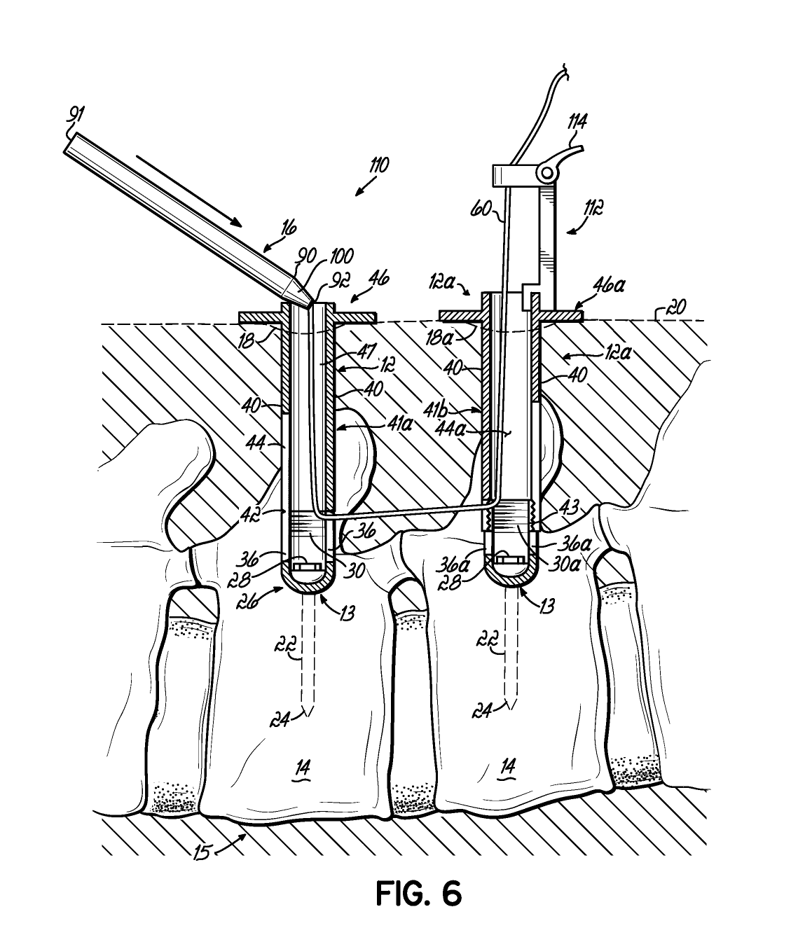

Coupling between the spine rod 16 and the cord member 60 may further take the form of a releasable coupling, such that, once the spine rod 16 has been positioned in its final configuration (as depicted in FIG. 6), the cord member 60 can be released and retrieved away from the spine rod 16. Alternatively, the coupling between the spine rod 16 and the cord member 60 may be a fixed coupling, such that, once the spine rod 16 has been positioned in its final configuration, a surgeon may sever the cord member 60, thereby leaving a portion of the cord member 60 as part of the final construct of the spinal fixation system 10.

With continued reference to FIGS. 4-5, the respective shapes of the spine rod 16 and lumens 47, 47a are such that the spine rod 16 can be received within each lumen 47, 47a and such that reorientation of the spine rod 16 can be achieved with relative ease. Reorientation of the spine rod 16 is similar to the orientation described above in regard to the rigid member 58. Thus, the illustrative slot 44 also facilitates reorientation by providing a space to receive the trailing end 91 of the spine rod 16, as the spine rod 16 is reoriented from travel in a first direction (i.e., down the lumen 47) to a second direction (i.e., between the two pedicle screw assemblies 12, 12a).

The shape of the spine rod 16 is further chosen such that it can travel between the lateral channels 30, 30a of the adjacent pedicle screw assemblies 12, 12a and against any resistance posed by tissue located between the two pedicle screw assemblies 12, 12a. To further facilitate travel of the spine rod 16 as described above, the leading end 90 of the spine rod 16 may include a taper 100 to reduce the area of initial contact between the spine rod 16 and surrounding structures such as tissue.

In one aspect of this embodiment, a tool or device (not shown) may be introduced through the lumen 47 to push the spine rod 16 and facilitate travel of the spine rod 16 between the lateral channels 30, 30a and through surrounding tissue. Similarly, one or more rigid or semi-rigid elements (not shown) can be attached to the cord member 60 between the handle 62 and the spine rod 16, to clear the path for the spine rod 16 through the tissue. Such rigid or semi-rigid elements make take on any suitable shape to facilitate travel of the spine rod 16. For example, and without limitation, these elements make take on a spherical, semi-spherical or conical shape.

With reference to FIG. 5, once the spine rod 16 has been positioned between the two adjacent pedicle screw assemblies 12, 12a, a set-screw driving tool 105 may be inserted through each lumen 47, 47a to transport and threadably fasten each set screw 50 against corresponding portions of the body of the spine rod 16, thereby securing the spine rod 16 in place within each of the polyaxial bodies 26, 26a of the adjacent pedicle screw assemblies 12, 12a. Moreover, and prior to securing the spine rod 16 in place as described above, a tool or device (not shown) may be inserted into either or both of the lumens 47, 47a to position the spine rod 16 in a desired final position. Such final position, for example, may be such that the leading end 90 of the spine rod 16 extends beyond the lumen 47a, as depicted in FIG. 5.

With reference to FIG. 6, in which like reference numerals refer to like features in FIGS. 1-5, an alternative embodiment of a rod installation assembly 110 includes a gripper member in the form of a lever device 112 suitably coupled to the flange 46a of the pedicle screw assembly 12a. The lever device 112 engages the cord member 60 such that engagement of an actuator 114 causes a pulling motion of the cord member 60. In one aspect of this embodiment, coupling of the lever device 112 to the flange 46a provides a fixed path and anchoring position for the cord member 60, as it is being is pulled.

In another aspect of the embodiments herein described, once the spine rod 16 has been positioned between the two adjacent pedicle screw assemblies 12, 12a and the set screws 34 correspondingly fastened, the flanges 46, 46a may be removed upwardly from the pedicle screw assemblies 12, 12a and the tabs 40 separated from corresponding polyaxial bodies 26. Thus, in the exemplary pedicle screw assembly 12, the tabs 40 thereof are broken along the frangible joints 42 and removed from the surgical site. Similarly, in the exemplary pedicle screw assembly 12a, the tabs 40 are unscrewed from the corresponding polyaxial body 26a to define the final construct of the spinal fixation assembly 10.

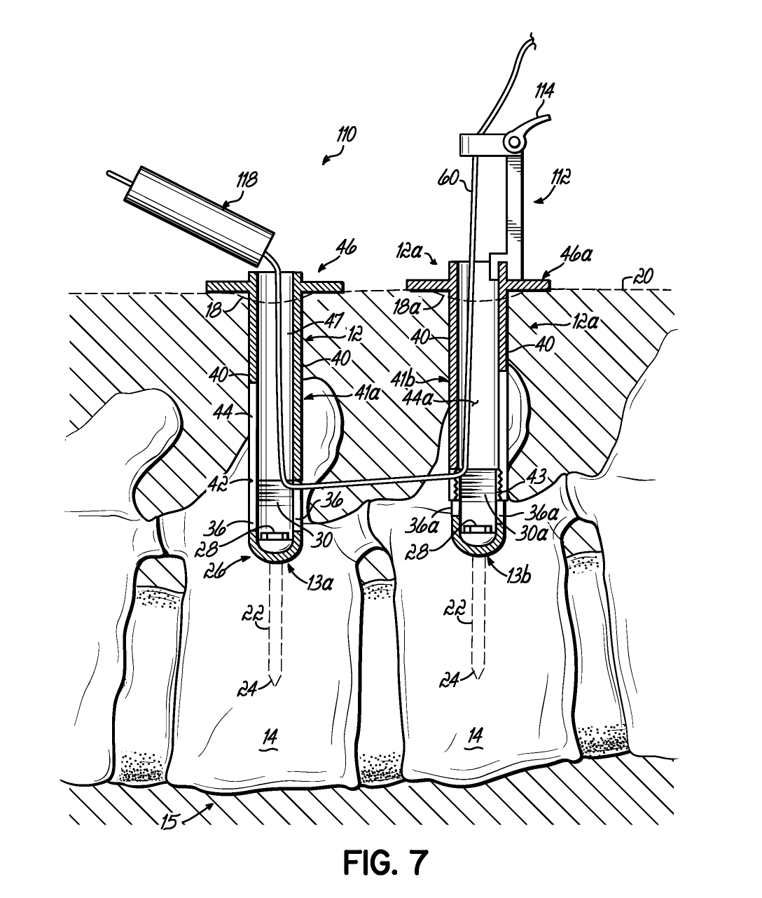

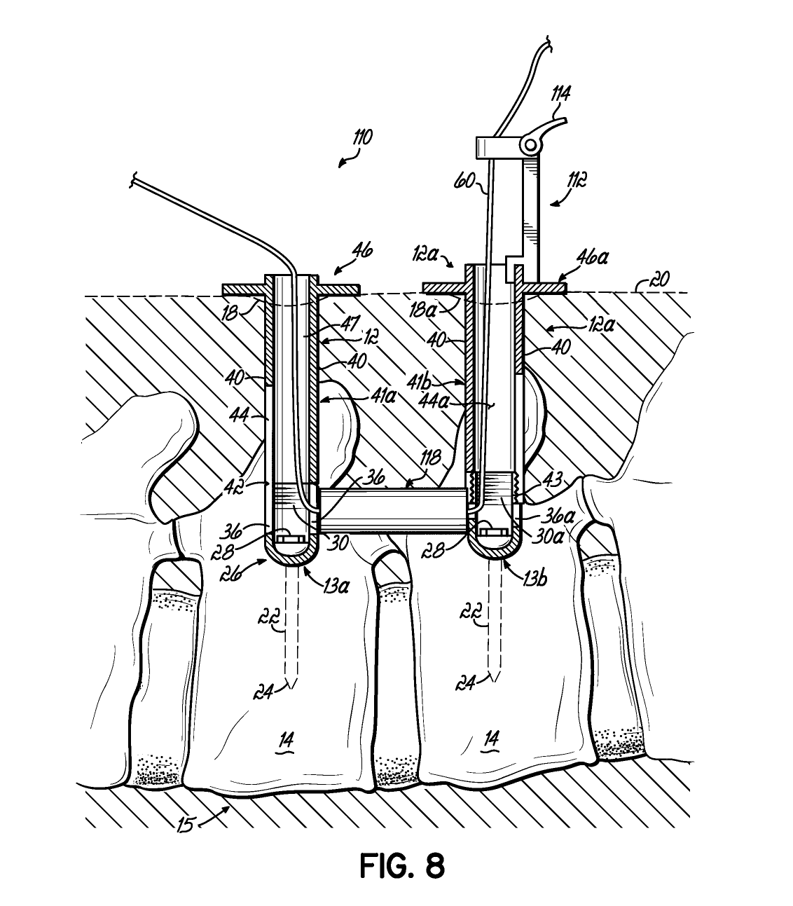

With reference to FIGS. 7-8, in which like reference numerals refer to like features in FIGS. 1-6, an alternative embodiment of a rod installation assembly 110 includes a actuator that is a tensioning member 114 suitably coupled to the flange 46a of the pedicle screw assembly 12a. In the embodiment of FIGS. 7-8, the flexible cord 60 is a cord similar to the cord that is used in the Dynesys® Dynamic Stabilization System available from Zimmer Spine of Minneapolis, Minn. A spacer 118, like the spacer used in the Dynesys® Dynamic Stabilization System, can be placed over a portion of flexible cord 60 extending out of incision 18 and pedicle screw assembly 12. The spacer 118 can be moved along the flexible cord 60 through a first tubular structure 41a to a location between the pedicle screws 13a, 13b as shown in FIGS. 7-8. The flexible cord 60 can then be fastened to a first pedicle screw 13a and the cord can be tensioned using the tensioning member 114. When reaching the desired tension, the flexible cord 60 can be fastened to a second pedicle screw 13b. Any excess of the flexible cord 60 can then be removed from the construct. In the embodiment shown, the remaining flexible cord 60 acts as the connecting element 16 between pedicle screws 13a, 13b.

The spacer 118 may be formed from polycarbonate urethane and the flexible cord 60 of the embodiment in FIGS. 7-8 may be formed from polyethylene-terephthalate. It will be recognized that various other materials suitable for implantation within the human body and for providing stabilization of the spine while maintaining flexibility may be used.

From the above disclosure of the general principles of this invention and the preceding detailed description of at least one embodiment, those skilled in the art will readily comprehend the various modifications to which this invention is susceptible. Therefore, it is intended for the invention to be limited only by the scope of the following claims and equivalents thereof.

What is claimed is:

1. A spinal fixation installation assembly comprising:
an elongate member including a trailing end and being configured for travel in a first direction along an elongate portion of a first bone anchor coupled to a spine and in a second direction between said first bone anchor and a second bone anchor coupled to the spine;
a cord member coupled to said trailing end of said elongate member; and
a connecting element having a leading end, said connecting element being coupled to said cord member at said leading end and being configured for travel in said first and second directions.

2. The installation assembly of claim 1 wherein said elongate member is a needle.

3. The rod installation assembly of claim 1 wherein the elongate portion is a tubular portion extending percutaneously from the spine.

4. The rod installation assembly of claim 1 wherein said cord member comprises at least one of a group consisting of a metal and a polymer.

5. The rod installation assembly of claim 1 wherein said connecting element further comprises a taper adapted to facilitate travel of said spine rod between the first and second bone anchors.

6. The rod installation assembly of claim 1 wherein said first direction is generally toward the spine.

7. The rod installation assembly of claim 1 wherein said connecting element is releasably coupled to said cord member.

8. The rod installation assembly of claim 1 wherein said elongate member is further configured for travel in a third direction along an elongate portion of the second bone anchor, said third direction being generally away from the spine.

9. The rod installation assembly of claim 8 further comprising a gripper member coupled to said cord member and adapted to pull said cord member in said third direction.

10. The rod installation assembly of claim 9 wherein said gripper member is further coupled to a percutaneous portion of the second bone anchor.

11. A spine fixation system comprising:
first and second adjacent bone anchors adapted to be coupled to a spine, said first and second bone anchors respectively including first and second lumens adapted to extend percutaneously from the spine;
a connecting element having a leading end and being adapted for fixation against said first and second bone anchors;
a cord member coupled to said leading end of said connecting element; and
an elongate member coupled to said cord member and adapted to guide said cord member within each of and between said first and second lumens, thereby guiding said connecting element within said first lumen and between said first and second lumens.

12. The spine fixation system of claim 11 wherein said elongate member is a rigid member.

13. The spine fixation system of claim 11 wherein said cord member comprises at least one of a group consisting of a metal and a polymer.

14. The spine fixation system of claim 11 wherein said connecting element further comprises a taper adapted to facilitate travel of said connecting element between said first and second bone anchors.

15. The spine fixation system of claim 11 wherein at least one of said first and second bone anchors is a pedicle screw assembly.

16. The spine fixation system of claim 11 wherein said cord member is releasably coupled to said leading end of said spine rod.

17. The spine fixation system of claim 11 further comprising:
a gripper member coupled to said cord member and adapted to pull said cord member along said second lumen.

18. The spine fixation system of claim 17 further comprising:
a flange on a percutaneous portion of said second bone anchor, wherein said gripper member is further coupled to said flange.

19. The spine fixation system of claim 11 wherein said first bone anchor further comprises:
a slot configured to receive a portion of said connecting element to thereby facilitate reorientation of said spine rod from a first direction along said first lumen to a second direction between said first and second lumens.

20. The spine fixation system of claim 11 wherein at least one of said first and second lumens is defined within an elongate portion removably coupled to a base portion of said first or second bone anchor.

21. The spine fixation system of claim 20 wherein said base portion comprises a polyaxial body of said first or second bone anchor.

22. The spine fixation system of claim 20 wherein said elongate portion is coupled to said base portion along a frangible joint.

23. A method of implanting a connecting element between first and second bone anchors coupled to a spine, the method comprising:
guiding an elongate member coupled to a first end of the connecting element in a first direction within a first elongate portion of the first bone anchor, the first direction being generally toward the spine;
guiding the elongate member in a second direction toward a second bone anchor; and
guiding the elongate member in a third direction within a second elongate portion of the second bone anchor, the third direction being generally away from the spine;
wherein the guiding steps induce travel of the connecting element in the first and second directions.

24. The method of claim 23 further comprising:
decoupling the elongate member from the connecting element.

25. The method of claim 23 further comprising:
securing the connecting element against each of the first and second bone anchors.

26. The method of claim 23 further comprising:
receiving the first and second elongate portions in a body of a patient respectively through first and second incisions in a patient's skin proximate selected vertebrae of the spine.

27. The method of claim 23 further comprising:
gripping the cord member proximate and percutaneous to the second bone anchor; and then
pulling the cord member in the third direction.

28. A method of implanting a spinal system between first and second vertebrae of a spine, the method comprising:
coupling first and second bone anchors to the first and second vertebrae;
providing percutaneous access to the first and second bone anchors through first and second tubular structures respectively coupled to the first and second bone anchors;
guiding an elongate member coupled to a flexible cord in a first direction within the first tubular structure, the first direction being generally toward the spine;
guiding the elongate member in a second direction toward the second bone anchor; and
guiding the elongate member in a third direction within the second tubular structure, the third direction being generally away from the spine;
placing a spacer over the flexible cord and guiding the spacer through the first tubular structure to a position between the first and second bone anchors.

29. The method of claim 28, further comprising the steps of:
securing the flexible cord to the first and second bone anchors forming a connecting element with the flexible cord; and
removing excess portions of the flexible cord.
